(19) United States
(12) Patent Application Publication
(10) Pub. No.: US 2016/0183803 A1
(43) Pub. Date: Jun. 30, 2016

(54) Video Voiding Device For Diagnosing Lower Urinary Tract Dysfunction

(71) Applicant: KING ABDULAZIZ UNIVERSITY, Jeddah (SA)

(72) Inventors: Hisham A. Mosli, Jeddah (SA); Nazeeh Alothmany, Jeddah (SA); John G. Webster, Madison, WI (US); Mehdi Shokoueinejad Maragheh, Tehran (IR); Rayan Alkashgari, Madison, WI (US)

(73) Assignee: KING ABDULAZIZ UNIVERSITY, Jeddah (SA)

(21) Appl. No.: 14/211,747

(22) Filed: Mar. 14, 2014

Related U.S. Application Data
(60) Provisional application No. 61/793,835, filed on Mar. 15, 2013.

(51) Int. Cl. A61B 5/00; A61B 5/20; A61B 10/00; A61B 7/04

(52) U.S. Cl. CPC A61B 5/0077; A61B 7/04; A61B 5/208; A61B 5/0013; A61B 5/7282; A61B 5/6889; A61B 10/007

(57) ABSTRACT

The shape of the urine stream can be used to measure both the flow rate and orifice geometry. The measurement of the shape of the urine stream can be a useful diagnostic tool for medical practitioners since it provides a noninvasive method of measuring urine flow rate and urethral dilation. A novel diagnostic visual voiding device (VVD) measures Symptoms of Lower Urinary Tract Dysfunction (LUTD) such as urinary incontinence. The VVD consists of a lightweight foldable booth with LED lights, microphone, video cameras on left side, top, face, and container that display stream height, width, angle, distance, face grimacing, container height and flow versus time. The VVD will improve the initial diagnosis of Lower Urinary Tract Dysfunction (LUTD) and also improve LUTD by providing subsequent diagnosis after treatment. It enables the patient to video and audio record urinary voiding in privacy. The video and audio recording hardware and software enables the video and audio recording to be sent in electronic form to the urologist at a location other than where the VVD is located.

### CROSS-REFERENCE TO RELATED APPLICATIONS

This application claims priority from U.S. Provisional Patent Application Ser. No. 61/793,835, filed on Mar. 15, 2013, the entirety of which is expressly incorporated by reference herein.

### FIELD OF THE INVENTION

The present invention relates to a urological disorders, and more particularly to a device and method for diagnosing a urological disorder.

### BACKGROUND OF THE INVENTION

Lower urinary tract dysfunction is common in both men and women, and the incidence and prevalence increase with advancing age (Dietz 2007). Symptoms of lower urinary tract dysfunction (LUTD) encompass all urinary symptoms including storage, voiding, incontinence, and post-micturition symptoms (Madersbacher et al 2004). Symptoms of LUTD are highly prevalent and occur in both genders to a similar extent, with 51% of men and 59% of women exhibiting storage symptoms; 26% of men and 20% of women exhibiting voiding symptoms; and 17% of men and 14% of women exhibiting postmicturition symptoms.

The impact and burden of symptoms of LUTD to individuals and to the nation are enormous. Those patients with symptoms of LUTD suffer considerable morbidity resulting in a significant decrease in quality of life for both the patient and his/her partner. According to the results from the National Institute of Diabetes and Digestive and Kidney Diseases (NIDDK) supported Urologic Disease in America project (http://grants.nih.gov/grants/guide/rfa-files/RFA-DK-11-026.html. incorporated by reference herein in its entirety), the diseases related to the prostate, only one of the organs that may contribute to lower urinary tract symptoms, cost 2.5 billion dollars to the nation in the year 2000 exclusive of outpatient treatments. A recent analysis of the National Health and Nutrition Examination Survey showed that when urinary incontinence was defined as “urine leakage during physical activity, before reaching the toilet and during nonphysical activity”, the prevalence is 51% in women and 14% in men aged 20 years old or older.

Many patients are unwilling to allow their physicians to observe them during voiding or at times of urinary incontinence in order for the treating doctor to make a judgment of the condition that would help in finding the correct solution for the problem. Further, patients lack the expertise to estimate and describe the magnitude of the force and caliber of their urine flow since they do not have a means to compare with others, some of them just don't observe their urine flow by nature such as women and others are not able to make this observation because of their shape, weight, age or illness (Pel et al 2002).

When the trained physician eye is allowed to visualize and examine the disorder, a solid conclusion can be made and the therapeutic decision is based on clear evidence, the power of vision. Traditional testing of voiding such as uroflowmetry, urethrography and urethral calibration do not include an actual visual examination of voiding by the clinician's eye (Chris et al 2011), They produce graphic traces, radiological images and impressions that are less helpful than watching the act itself (Shafer et al 2002). Furthermore, the confirmation of clinical diagnosis of some voiding disorders such as stress urinary incontinence depends solely on visual inspection.

The devices in current use to study voiding actually measure urine flow or volume per unit time or the so called uro-flow-rate (rate of urinary flow), time of voiding, average flow rate over the total voiding time and so on (Reynard et al 1996). Among those, the most important parameter is the maximum flow rate (Q-max.) or the peak flow rate defined as the highest point on the curve the flow reaches during the period of voiding (Schafer et al 2002). This is purely physics and recorded by a device that depends on only one of the physical properties of the voided urine, that is weight and the speed of its accumulation (Addla et al 2010). It records the measurement at the end of the urine stream or column at a point the stream may have already been slowed, branched, bifurcated or turned into a spray rather than a one solid line. Therefore, other more important clinical characteristics of the act of voiding and the nature of the voided urine are not observed. Clearly, to the urologist, learning about the force of the urine during its natural flow as visually observed and noting its direction and caliber is far more important especially in certain medical circumstances such as obstructed flow of urine, deviated stream (Bloom et al 1985), stress urinary incontinence and conditions associated with interrupted stream such as detrusor sphincter dysenergia (Riehmann et al 1998).

Today's technology allows for this opportunity by utilizing miniature devices to provide a clear view to the treating doctor to make the needed diagnosis (Boci et al 1999, Caffarel et al 2007, Cafferal et al 2006, and Currie 1998). However, to date, even with this available technology there still is an unmet need to find a better way to examine the urine flow during the act of voiding to estimate its direction, force and caliber (Chris et al 2011 and Matthew et al 2011) and hereby provide an accurate diagnosis of the condition of the patient.

As a result, it is desirable to develop a reliable device and method of use for accurately diagnosing the symptoms of lower urinary tract dysfunction in individuals that may also be used in a manner that preserves the privacy of the patient to accurately and non-invasively determine the extent of the condition and the progression of the treatment of the condition after initial diagnosis.

### SUMMARY OF THE ENVENTION

According to one aspect of the present disclosure, a Visual Voiding Device (VVD) is provided that can improve the initial diagnosis of LUTD and also improve LU ID by providing subsequent diagnosis during and after treatment of LUTD. The VVI) device provides a private environment in which the patient can perform the required voiding function to alleviate any issues in function performance on the part of the patient and is complementary in the primary diagnosis of Lower Urinary Tract Dysfunction (LUTD) rather than having a strictly therapeutic role. By helping to make the correct diagnosis and evaluating the degree of LUTD severity, the device is of clinical importance especially in the documentation and further monitoring of the progress of this condition.

In one embodiment of the device, the device includes an enclosure having a video device therein capable of recording the urination by an individual which can then be processed using a suitable computing device to measure the shape of the urine stream. The measurement of the shape of the urine stream is a useful diagnostic tool for medical practitioners since it provides a noninvasive method of measuring urine flow rate and urethral dilation. The novel diagnostic visual voiding device (VVD) measures Symptoms of Lower Urinary Tract Dysfunction (LUTD) such as urinary incontinence. The VVD includes a lightweight foldable booth with LED lights, microphone, video cameras on left side, top, face, and container that display stream height. width, angle, distance, face grimacing, container height and flow versus time with a high level of accuracy. The VVD improves the initial diagnosis of Lower Urinary Tract Dysfunction (LUTD) and also improves the treatment of LUTD by providing subsequent diagnosis after treatment. It enables the patient or physician to video record urinary voiding in privacy. The video recording hardware and software enables the video recording to be sent to/front a remote location where the patient is diagnosed or treated to the urologist for further analysis.

According to another aspect of the present invention, this device will also obviate the need for more invasive testing such as radiological examination and endoscopy. It can be used for men, women and children at different age groups. It is more convenient for both the patient and the doctor to observe the act of voiding remotely using a monitor while the patient is seated comfortably in privacy.

Numerous other aspects, features and advantages of the present invention will be made apparent from the following detailed description taken together with the drawing figures.

### lBRIEF DESCRIPTION OF THE DRAWING FIGURES

The drawings illustrate the best mode currently contemplated of practicing the present invention.

In the drawings:

FIG. 1 is an isometric view of one embodiment of a device constructed according to the present invention, illustrating a patient standing ready to urinate into a collection element;

FIG. 2 is a side plan view of the device of FIG. 1 with a stream to be analyzed;

FIG. 3 is a side plan view of the perspective of one of the cameras used in the device of FIG. 1;

FIG. 4 is a side plan view of the perspective of another of the cameras used in the device of FIG. 1;

FIG. 5 is a schematic view of a decision tree for the development of particular construction of the device of FIG. 1;

FIG. 6 is a schematic view of a display used with the device of FIG. 1 showing the pictures from the respective cameras of the device;

FIG. 7 is a perspective view of a volume measuring cylinder used with the device of FIG. 1 as viewed by a camera of the device of FIG. 1;

FIG. 8 is an isometric, broken-away view of the floating object used to track the level of urine in the collection member of FIG. 7;

FIG. 9 an isometric view of a second embodiment of a collection element used with the device of FIG. 1;

FIG. 10 is a schematic view of a third embodiment of a collection element including a scale used with the device of FIG. 1;

FIG. 11A and 11 B are schematic views of fourth and fifth embodiments of collection elements used with the device of FIG. 1;

FIG. 12 is a perspective view of the collection member of FIG. 9 showing the level of dyed urine as it rises up the collection member as viewed by a camera of the device of FIG. 1;

FIG. 13 is a perspective view of a sample flow stream as viewed by a camera of the device of FIG. 1 including data about the flow stream;

FIG. 14 is a schematic view of a flowchart for the development of the computational process for utilization of the data and video obtained by the use of the device of FIG. 1;

FIG. 15 is a graph of flow rate vs. time with and without wave effect filtering;

FIG. 16 is a schematic view of a EM flow meter used in comparison test with the device of FIG. 1;

FIG. 17 is a graph of the non-interrupted flow rate vs, time using the EM flow meter of FIG. 16 and the Scale and Video methods suing the device of FIG. 1; and

FIG. 18 is a graph of the interrupted flow rate vs. time using the EM flow meter of FIG. 16 and the Scale and Video methods using the device of FIG. 1.

### DETAILED DESCRIPTION OF THE DISCLOSURE

With reference now to the drawing figures in which like reference numerals designate like parts throughout the disclosure, a first embodiment of the VVD device 10 of the present disclosure is illustrated that includes a collection element or funnel 12, a number of video cameras 14, which in the illustrated embodiment are four (4) in number but can be one or more as desired, a privacy booth 16 disposed around the collection element 12, which can be a toilet, among other suitable elements, and on which the cameras 14 and a number of lights 18, such as

LED lights, can be mounted, and a graduated, partitioned cylinder 20, though other constructions of the device 10 are also contemplated as being within the scope of the present disclosure.

The following are examples of the clinical application of the use of the VVD device 10 illustrating its use in the initial diagnosis, documentation of the diagnosis for later review, and treatment management of LUTD, therefore helping in improving this condition.
b 1. In the initial confirmation of the presence of LUTD as claimed by the patient P (diagnosis), there are 3 types of Urinary Incontinence (UI):
   a. Total (continuous) urine incontinence UI e.g. post-prostatectomy incontinence: the well hydrated patient P will he asked to step into the device 10 and then record for a short period of time of 5 to 10 min according to the tolerance of the patient. The urine should he visualized and recorded by the video cameras 14 to be continuously leaking from the urethral meatus. At the same time leaking urine is digitally calculated by tracking the urine-motion using the recorded video, and an attempt is made to collect and measure this amount of urine for comparative purposes. The 24 h amount of leaking of urine can be estimated in this manner using the device 10 to speed up the initial diagnosis of the condition of the particular patient P. This is in contrast to the currently used method for diagnosis and estimation of the severity is the 24 h pad test., i.e. collecting the urine that leaks in the diaper worn for 24 h then the pad is weighed and the amount of urine is estimated.
   b.Stress urine incontinence (SUI): in this type the urine leak does not show unless there is a physical increase in the intra-abdominal pressure. Therefore, the well hydrated patient P (female or male)—preferably with full bladder—will be asked to be placed in front of the device 10 (best position is lithotomy position i.e. legs up on the stirrups). The device 10 will be turned on in order t record the event, and then the patient P will be asked to cough or bear-down (strain) and the amount of urine that leaks can be momentarily recorded on the cameras 14. The event recorded by the cameras 14 can be used to estimate the volume of the released urine according to any known manner, such that the urine volume estimated and the duration of incontinence is also noted. This can be repeated as many times as deemed necessary for the physician to make an accurate diagnosis. This use of the device 10 is in contrast to the currently used method to confirm the diagnosis is by visual inspection usually in the cystoscopy suite without proper documentation of the event. The severity is estimated according the patient's story (history) of the number of pads she or he had to change per day, and whether the pads were damp, wet or soaked.
   c. Urge urinary incontinence (urge UI ): the VVD 10 can be used in combination with conventional urodynamic testing in cases of urge UI. This is in contrast to the current method of diagnosis which is heavily dependent upon the patient's history and the picture seen in the urodynamic testing (cystometrogram=CMG), no current visual documentation of the events of urge incontinence were ever studied and documented on camera.
Documentation via recorded video, audio and associated data collection of the type and degree of UI as mentioned above for the determination or diagnosis of each of the various types of UI and for use in comparison regarding future data and video recorded on the same patient(s).
3. In the follow up monitoring of the various types of treatment:
   a. Physiotherapy and pelvic floor exercises: the VVD 10 will help in monitoring the progress of Ul following this type of therapy by subsequent evaluation of the patient using the device 10 in any of the manners described above for the use of the device 10.
   b. Medical treatment of the VVD will help in monitoring the effectiveness of medical treatment by subsequent evaluation of the patient using the device 10 in any of the manners described above for the use of the device 10.
   c. Surgical treatment of Ul: the VVD will help in monitoring the success or failure of surgical management, the degree of improvement of UI can be assessed and documented following this type of therapy by subsequent evaluation of the patient using the device 10 in any of the manners described above for the use of the device 10.

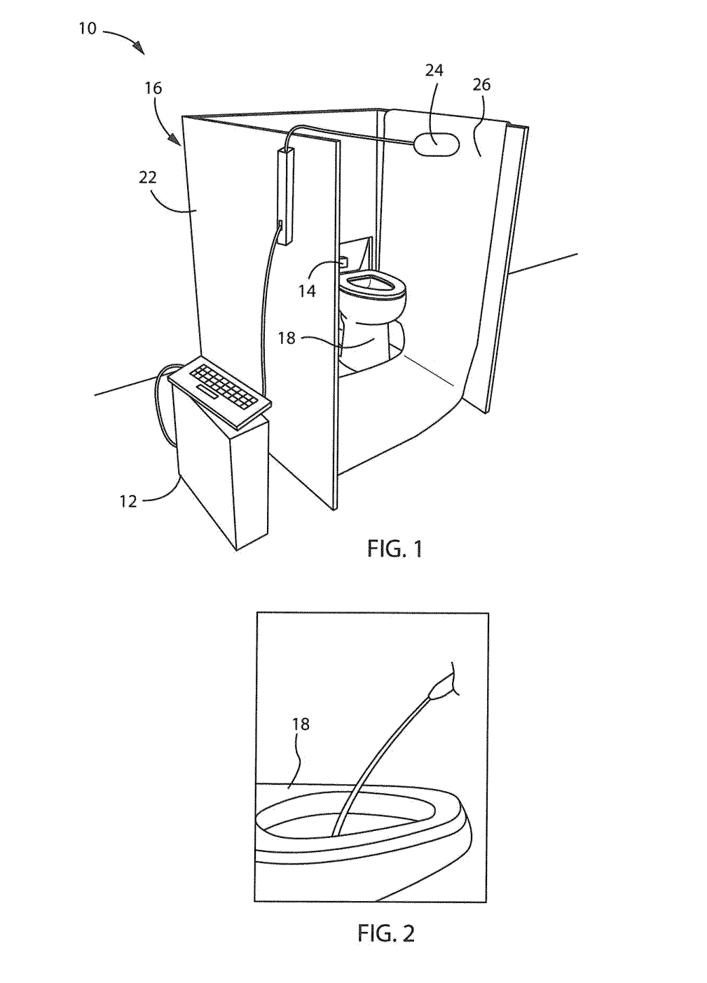

The booth 16 shown in FIG. 1 is used in one embodiment of the device 10 which uses light hollow plastic walls 22, LED lights 18 mountable as desired to the walls 22, and which is readily disassembled and/or foldable for storage. The shape of the urine stream as recorded in the device 10 can be used as an indicator of both the flow rate and orifice geometry for use in making the diagnosis or follow up evaluation of the patient P. The measurement of the shape of the urine stream can be a useful diagnostic tool for medical practitioners since it provides a noninvasive method of measuring urine flow rate and urethral dilation. The plastic walls 22 of the U-shaped booth 16 are hinged for folding and storage. The side camera 14 views a flat black wall 24 or a black portion 24 of wall 22 forming a portion of the booth 16. LED lights 18 mounted on the booth 16 where desired illuminate the stream 26 to provide best contrast. Microphones (not shown) can also be added to the device 10 by placing the microphones on the walls 22 where desired to record audio to go along with the video recorded by the cameras 14. This audio can be subsequently processed simultaneously with or separately from the video in the various computer or other electronic device operably connected to the device 10 and its components for this purpose.

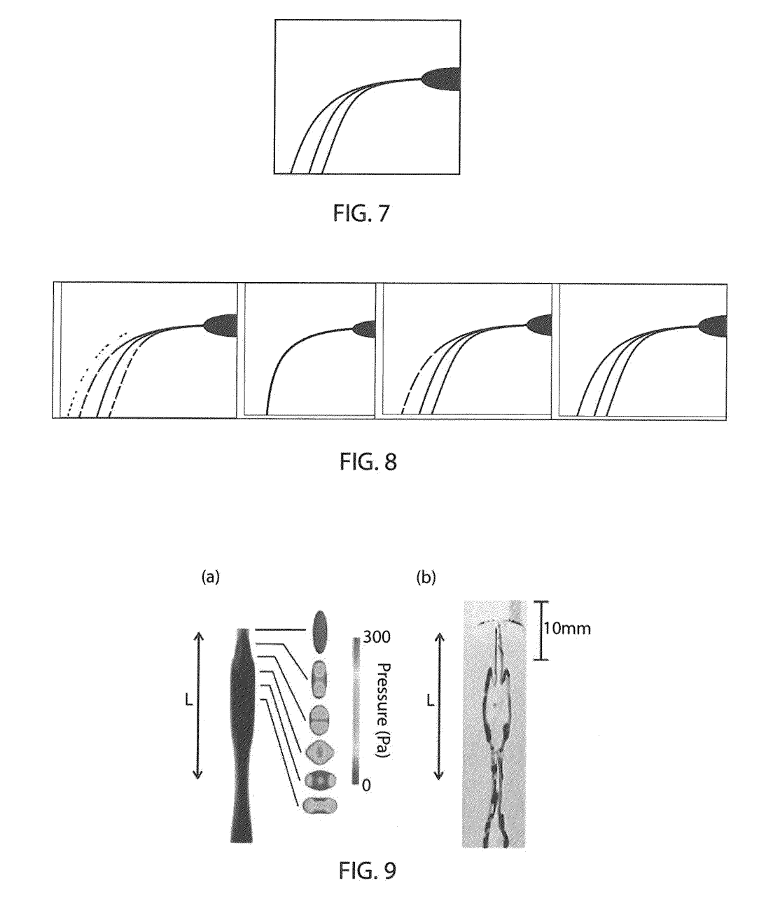

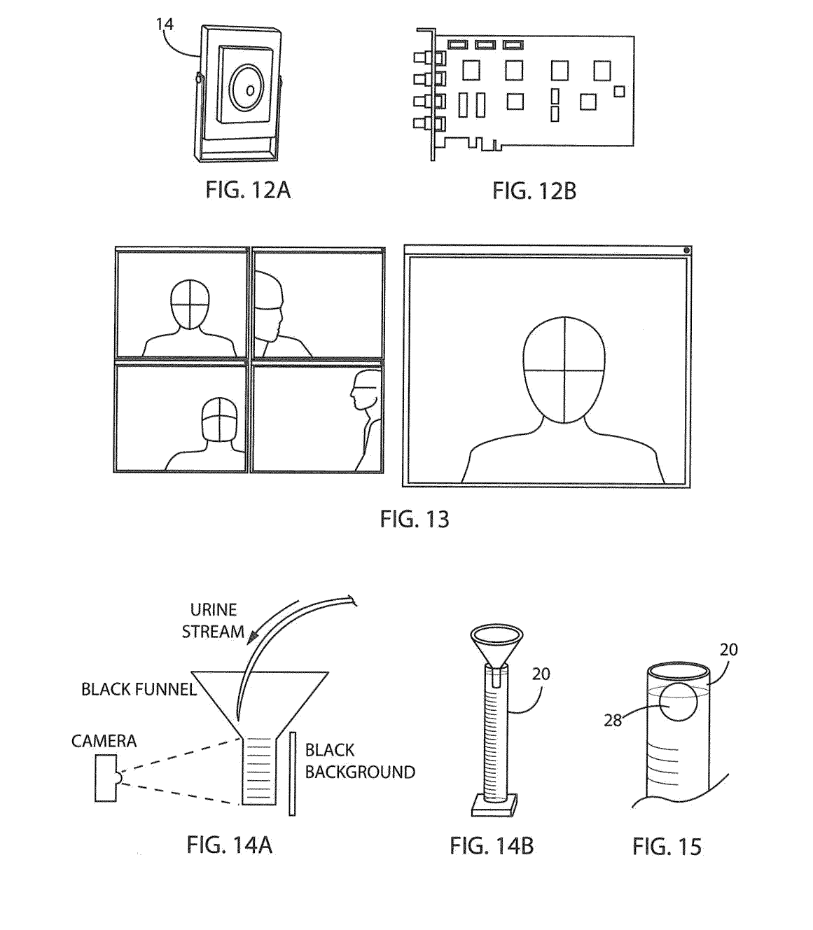

FIG. 2 shows the good contrast when the camera 14 is facing a flat black wall 24 or black portion 24 of wall 22 and the LED illumination/light 18 is not in the camera viewing angle. This system or device 10 is useful in diagnosing male Lower Urinary Tract Symptoms (LUTS). Formulas and computer algorithms determine and use: 1) stream 26 cross section; 2) initial angle; 3) distance traveled to calculate flow versus time. Wheeler has developed formulas to determine flow versus time as illustrated in Wheeler A. P. S., Morad S. Buchholz N, Knight M M. The Shape of the Urine Stream—From Biophysics to Diagnostics PLOS ONE, October 2012|Volume 71Issue 10 e47133, expressly incorporated by reference herein in its entirety. Wheeler's article shows one way of measuring flow. The VVD 10 measures urine stream 26 angle and velocity and provides an acceptable estimation of the urination flow rate. A graduated cylinder 20, as shown in FIGS. 7 and 12, is used for recording the flow versus time. A black funnel 12 directs the stream 26 into the graduated cylinder 20. One of the cameras 14 is aimed at the cylinder 20 to record the movement of a plastic float 28 in the cylinder 20 (FIG. 7). or the level of dyed urine entering the cylinder 20 (FIG. 12) to record the volume versus time, from which flow versus time is plotted.

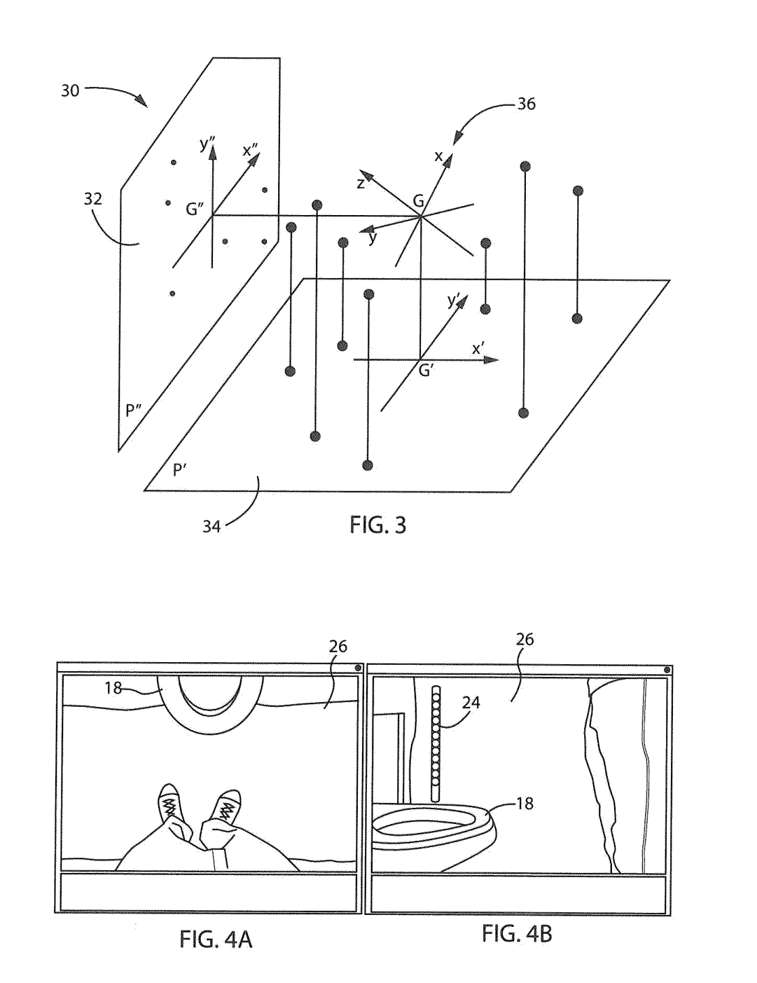

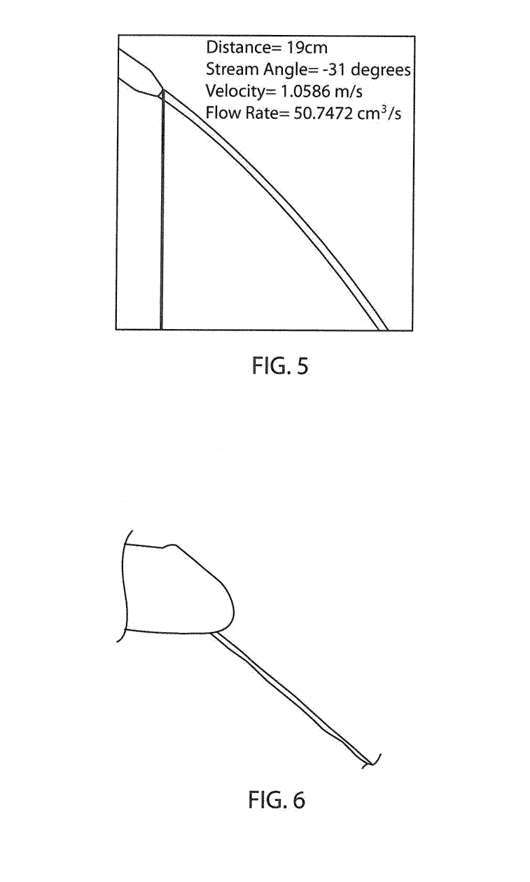

FIGS. 3 and 4 show views from the top and side cameras 14 of the device 10 in the frame for 3D video capturing. FIG. 4 is a schematic view from the top camera 14 showing the funnel 12 and volume in front of the torso. FIG. 3 shows the view of the side camera 14 shows subject standing on the right and LED lights 18 on the left out of the field of view of the camera 14.

To determine the proper set up for the device 10, development requires various computer hardware and software 100, including frame-grabber video processing software, electronic storage medium for storing and processing the video recorded by the cameras 14 and transmission software, as well as and hardware 102 for the booth 16, including the cameras 14 compatible with the software 100, walls 22, toilet or funnel 12, and graduated cylinder 20, which are known in the art and can be selected as desired, such as by using the decision tree of FIG. 6.

In the embodiment illustrated in FIGS. 1-4. the U-shaped booth plastic walls 22 are hinged for folding and storage. The side camera 14 views a flat black wall or black portion 24 of wall 22. The cameras 14 used in this embodiment were a Logitcch c920, which is a digital full HD 1080p pro Webearn, with 20-step autofocus lens. Three LED lights 18, such as G-LUX series 8 Watt LED Spot Light, are mounted to the walls 22 to illuminate the stream 26 against the black wall 24 to provide best contrast for the cameras 14. Two LEDs 18 are mounted on the wall 22 facing the patient P (above and below the urine stream line of action). A third LED 18 is mounted at the top of the right side wall 22, shedding light from a steep downward orientation/trajectory towards the funnel 12. The combination of these three LED 18 placements shedding light from three different angles results in complete illumination of the urine stream 26, ensuring high contrast and visibility.

In operation, the patient P stands in front of/within the device 10 and urinates. The urine is guided by the large funnel 12, which can be from 4 to 24 inches in diameter at the upper end to ensure capture of the entire flow, leading it to the cylinder 20. The urination is captured from two angles using two cameras 14 (top and side). A third camera 14 near the bottom the booth 16 captures the filling process of the cylinder 20 in order to enable computation of the instantaneous flow rate, max flow rate, total voiding time, and total voided volume. The fourth camera 14 captures the facial expressions of the patient P since they help the physician to determine the smoothness of the urination process for the patient P.

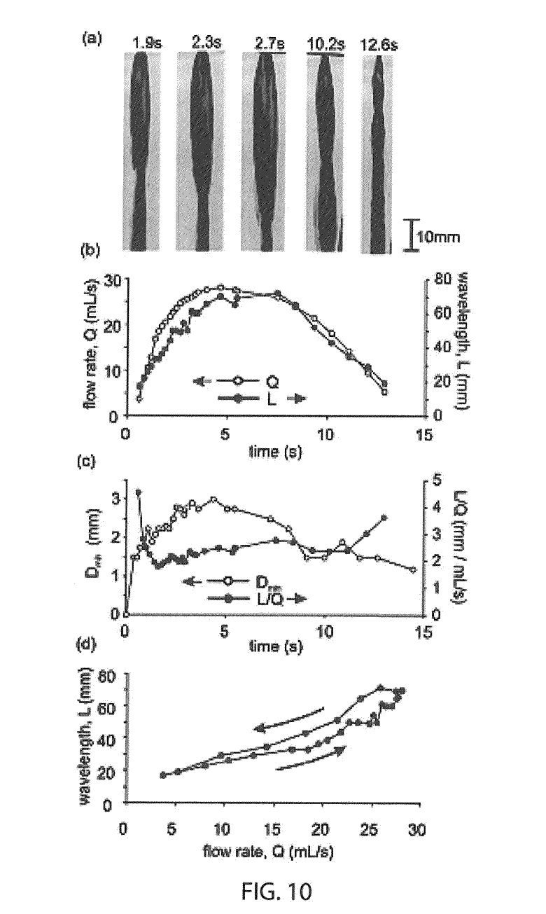

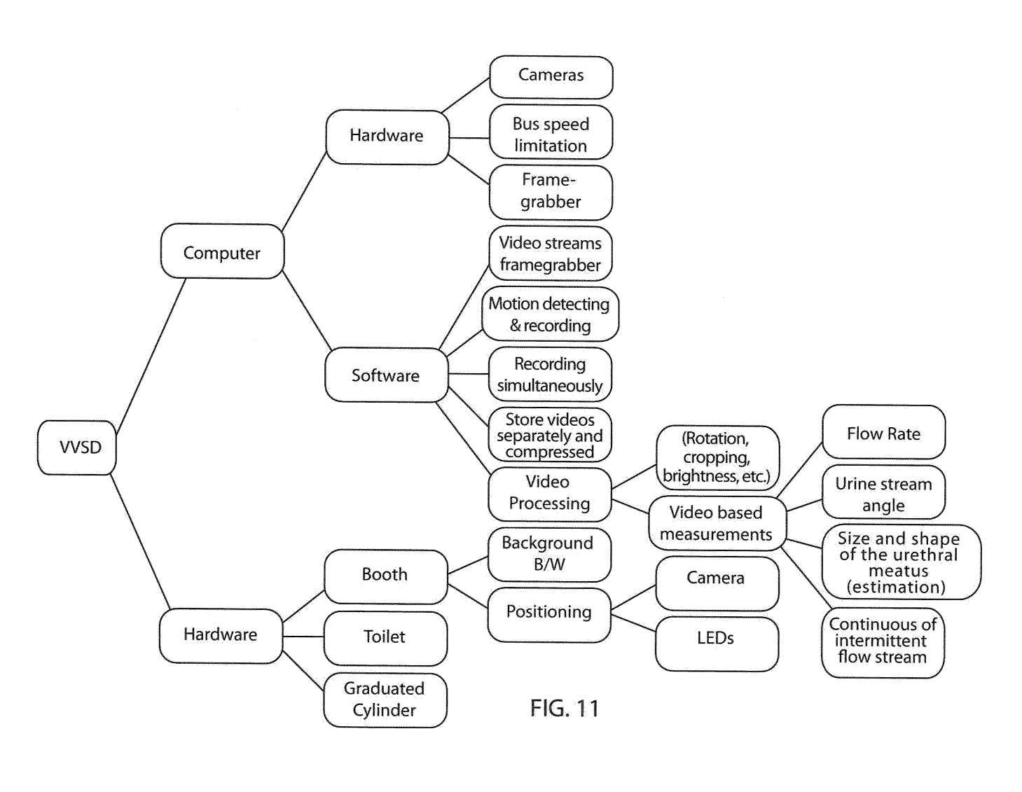

The device 10 also has many variations in its construction that can be utilized. For example, FIG. 6 shows a single monitor 150 used with divided screens where each of the cameras 14 in FIG. 1 displays its view in a corresponding segment of the monitor 150, and the screen segments assigned for future use will display flow graphs and data being generated by the device 10. FIGS. 7 and 8 show a plastic float 28 that can be colored or uncolored disposed in the cylinder 20 that provides good contrast for video recording of liquid level in the cylinder 20. The camera 14 directed at the cylinder 20 captures the image of the plastic float 28 on top of the liquid in the cylinder 20 with good contrast. FIG. 9 shows a commode chair 160 that could be slid above the cylinder 20 for situations requiring the patient to be in a sitting position. This embodiment of the system 10 would be convenient to measure both male and female urinary incontinence (Up. FIG. 10 shows an electronic scale 50 positioned under the cylinder 20 beneath the funnel 12. Using an electronic scale 50 to weigh the liquid in the cylinder 20 would permit measurement of volume and flow versus time using the device 10 within a regular or portable toilet 18. Then a raised platform (not shown) would not be required. This system 10 would also be convenient to measure both male and female urinary incontinence (UI). FIG. 11 B shows a female spread leg toilet seat 170 to yield good contrast of the urine stream. For females, in place of the mutual toilet seat 180 shown in FIG. 11A, a spread leg toilet seat 170 is designed to be comfortable with the legs spread. Urine flows into a black funnel 12 to be weighed by an electronic scale 50, as shown in FIG. 10, The back-lighted camera 14 from above videotapes the stream with good contrast against the black funnel.

As shown in Table 1, the benefits of the use of the VV D 10 are shown in comparison with prior art diagnosis tools.

TABLE 1
Uroflowmeters versus Visual examination
versus VVD (our new device)

| Uroflowmeters | Visual examination | VVD |
|---|---|---|
| Blind, unseen | Visual, seen by | Visual, seen by expert eyes |
| | expert eyes | |
| Accurate | Estimation only | Accurate (97.7 ± 4.5%) |
| Capacity: volume | Caliber | Capacity: volume |
| measured in ml | | measured in ml/cc and |
| or cc | | Caliber |
| Flow measured as | Direction | Flow measured as ml per |
| ml per second | | second |
| Influenced by other | Shape or form | Direction |
| factors such as | | |
| straining, positioning, | | |
| and environment | | |
| | Continuous versus | Shape or form |
| | interrupted | |
| | | Continuous versus |
| | | interrupted |

In using the device 10, the video recording of the patient's stream live and inspecting it by the physician is much more realistic, practical and convenient especially as the recorded video and any accompanying analysis can be sent in electronic form, such as by e-mail over the Internet, to the urologist who can be at a location different from where the device 10 is located.

The device 10 and method can be used alone or in conjunction with the traditional urodynamic study devices to form a useful combination to study the act of voiding from all its aspects.

In operation, this device 10 has certain objectives. One is to document the urine voiding process by capturing the urine stream from at least two angles (top and side) using the cameras 14. The measurement of the shape of the urine stream is a useful diagnostic tool for medical practitioners since it provides a noninvasive method of measuring urine stream aspect and urethral dilation.

Another objective is to extract parameters that would benefit the physician in making better diagnosis and decisions, such as the Maximum (Peak) Flow Rate, Average Flow Rate, Flow Rate, and Total Voided Volume. In order to measure the flow rate the cylinder 20 is set so the urine accumulates up the cylinder 20 while a third camera 14 monitors the process. However, urine can sometimes be clear in color, which makes it very challenging to be traced using a camera. To alleviate this problem, as shown in FIG. 12, a colored dye (10-15 drops) is added at the bottom of the cylinder 20 prior to the voiding process to color the urine collected in the cylinder 20 and provide greater contrast within the cylinder 20.

In an example of how to use the data and video obtained by the device 10, FIG. 13 is a side elevation view of the representative urine stream used to measure stream angle and velocity using the device 10, and the resulting determination of the urination flow rate. The VVD 10 tracks a continuous and/or non-continuous stream and determines the tip of the body portion. In particular, FIG. 13 is a sample frame from the side camera 14 with observed and calculated information from a water stream used to calculate stream flow rate.

In order to measure/calculate the total voiding time, e.g., in seconds, in the device 10 a third camera 14 is positioned below the mock toilet lid/funnel 12 for recording the flow versus time. The voided urine is collected in the clear cylinder 20 and its volume is measured by the third camera 14. The average voiding time (total urine volume divided by total voiding time in seconds) can then be determined using the information from the various cameras 14 and the cylinder 20.

In one embodiment. FIG. 14 shows the process for utilizing the data obtained by the device 10. Initially, first the raw data are loaded as a structure of frames through video acquisition. Each frame is then cropped to only show the middle part of the cylinder 20. Contrast is enhanced through whitening of anything that is not the color of the dye and blackening of the dyed water/urine. After that a tracking algorithm tracks the level of water/urine in the cylinder 20 and marks it on each slide. Volume is measured at each frame and flow is calculated by differentiating the volume over time. An averaging algorithm smooths the flow graph by detecting noisy positive and negative peaks in the graph and averaging them to plot a point half way between them. Both the flow graph and instant flow at each frame are embedded in the video to provide a deeper insight by allowing the physician to track the flow instantaneously as the patient urinates.

Suitable software has been developed using MATLAB to analyze the recorded video and calculate the real-time flow rate, the maximum flow rate, and the average flow rate, though other software is also capable of being used for this analysis. The software can also be adapted to analyze any audio that is recorded using one or more microphones disposed on the device 10, such as on one or more of the walls 22.

The software starts by loading the video frames and isolating the cylinder 20 from its surroundings. It then isolates the colored water/urine from its white background for optimum contrast. After that the software tracks the level of water/urine as it accumulates in the cylinder 20 and calculates the instant flow rate, maximum flow rate, average flow rate and total volume. One of the phenomenon that was noticed is that as the water/urine accumulates in the cylinder 20 it hits the water/urine surface causing a “wave effect”. One approach to solve this problem was to guide the water to slide down the inside cylinder wall, such as by using a divider 80 (FIG. 1) positioned within the cylinder 20. The result was a significant reduction in the “wave effect”, but nevertheless it was still present. And so a new filtering technique was developed as a counter measure to this phenomenon where the crest and trough of each wave are used to cancel each other erasing the existence of the wave (see FIG. 15). The result is then embedded within the video to provide a clear view of the water accumulation, the instant flow rate, the maximum flow rate, the average flow rate, and flow chart tracing the flow in real time.

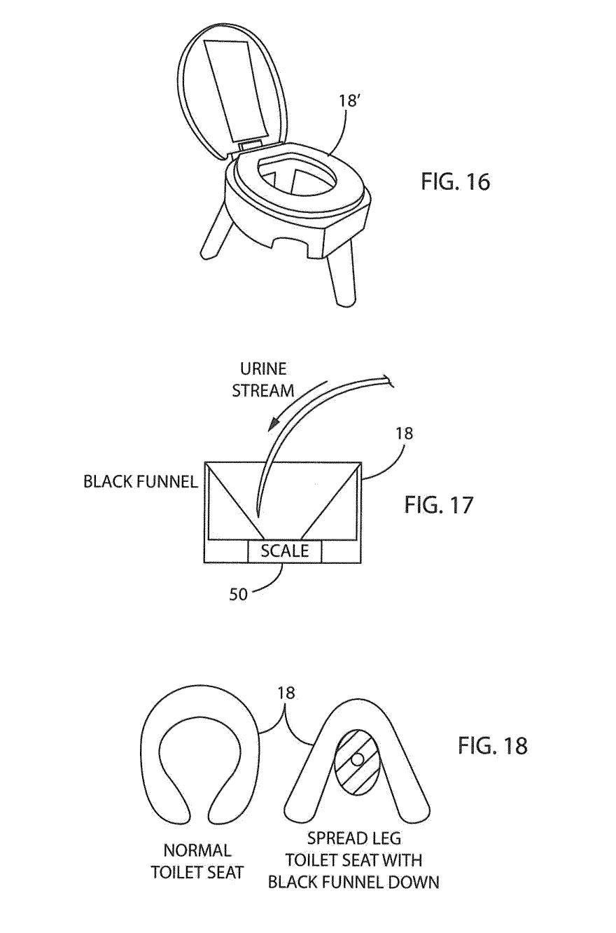

Method validation is the process that we used to confirm that the analytical and computational procedure employed for our video processing was suitable. Results from method validation were used to judge the quality, reliability and consistency of analytical results. For our system validation we used the Carolina Medical Measurements electromagnetic (EM) flow-meter Model 501D), shown in FIG. 16 at 200. In this device 10′, a balloon 302 filled with water is used to simulate the function of a human bladder. The balloon wall puts pressure on the water inside it, squeezing it out through a tube 304 that simulates the urethra. Saline was passed through the extravascular ¼ inch EM flowmeter Model EP300A ¼ as it departed the saline reservoir to calibrate our system that measures urinary flow. In addition, the cylinder 20 for the device 10, 10′ was positioned on a scale 50 (e.g., as in FIG. 10) that measured the weight of the stream collected over the time the stream was active for a Scale method used as a second method of comparison of the results obtained from the device 10, 10′.

FIG. 17 compares flow versus time as measured by the gold standard EM flow meter and our Video and Scale methods of measuring real flow from a human. Further, FIG. 18 shows the alternative pattern of mock, interrupted urination provided to examine the V VD 10 response in comparison with the EM flow meter and Scale methods. These show that the VVD 10 can detect the start and ending point of each voiding period. The maximum flow rate and the overall shape of the voiding periods are relatively close together. The good response from the video data from the device 10 therefore provides enough information for physicians to make effective and accurate diagnoses of LUTD in patients that have utilized the device 10. In particular, Table 2 shows that:
Compared to the standard EM flowmeter, the peak flow is 98.3 ±0.7%.
Compared to the standard EM flowmeter, the time delay is 1.08 ±0.6 s.
Compared to the standard EM flowmeter, the total volume is 97.7 ±4.5%.

TABLE 2
Comparison of test results obtained from
VVD, scale, and EM flowmeter.

| | | | | Total Voided |
|---|---|---|---|---|
| | Max Flow | Average Flow | Total Voiding Time | Volume |
| | (mL/s) | (mL/s) | (s) | (mL) |
| Video 1 | 35.47 | 8.16 | 23.83 | 194.45 |
| Scale 1 | 35.12 | 9.11 | 20.20 | 184.02 |
| EM 1 | 35.80 | 8.82 | 21.85 | 192.72 |
| Video 2 | 35.60 | 23.79 | 4.1 | 97.54 |
| Scale 2 | 39.07 | 20.57 | 4.60 | 94.62 |
| EM 2 | 36.37 | 24.96 | 3.81 | 95.10 |
| Video 3 | 40.7 | 21.47 | 6.83 | 146.64 |
| Scale 3 | 42.07 | 20.15 | 6.4 | 128.96 |
| EM 3 | 41.24 | 25.95 | 5.87 | 152.33 |
| Video 4 | 36.78 | 14.89 | 6.83 | 101.70 |
| Scale 4 | 33.52 | 15.32 | 5.8 | 88.86 |
| EM 4 | 37.76 | 19.53 | 5.72 | 111.71 |

The main function of the device is unique, has several important clinical values that have not been described before in any other currently available devices. The video-based Visual Voiding Study Device 10 is expected to be multitask to simultaneously perform the following:
1. Defining the exact direction of the urine flow in relation to the vertical axis of the human thorax i.e. deviation to the right or left and in relation to the direction of the flaccid penis i.e. upwards and downwards and determination of the degree of the angle of deviation i.e. 15, 20, 30 up to 90 degrees deviation. The same concept can be applied on the body of the erect penis where one can study and document the direction and degree of penile angulation in eases of Peyronie's disease and congenital penile curvature (Golomb et al 1992).
2. Recognition of fluid spraying, splitting and branching (bifurcationitrifurcation) of the urine stream.
3. Measure/calculate the total voiding time in seconds accurately. The voided urine will be collected and its volume will be measured. The average voiding flow (total urine volume divided by total voiding time in seconds) will be determined (Nitti 2005).
4. Determination of the distance of urine expulsion (Nitti 2005).
5. Recording, documentation and playback of momentarily loss (leak) of urine during coughing, sneezing and laughing, a condition known as stress urinary incontinence in both males and females.
6. Recording, documentation and playback of momentarily prolapse of pelvic organs during coughing, sneezing and laughing in female patients.
7. Recording the facial expressions of the patient to determine whether pain is involved or not.

The VVD 10 provides more useful diagnostic information using lower cost components than existing commercial flowmeters (De La Rosette et al 1996 and Greenwell et al 2004).The new proposed device will be very useful in the diagnosis of a number of urological disorders such as: Benign Prostatic Hyperplasia (BPH) (noncancerous enlargement of the prostate, which blocks urinary flow) (Jonas and Hofner 1996, Neal 1997, Wasson et al 1995) and Wiygul and Babyan 2009), urinary incontinence mainly stress urinary incontinence (undesired urinary flow when coughing), congenital anomalies that involve the bladder and the urethra (urinary tube) (Steenkamp et al 1997), neurogenic bladder (disease of the central nervous system or peripheral nerves involved in the control of urination) overactive bladder (sudden, involuntary contraction of the muscle in the wall of the bladder) and sonic gynecological conditions (Nitti 2005).

The following references are expressly made part of the disclosure of this application and are expressly incorporated by reference herein in their entirety:
1. Schafer W, Abrams P, Liao L M, Mattiasson A, Pesce F, et al. (2002) Good urodynamic practices: Uroflowmetry, filling cystometry, and pressure-flow studies. Neurourology and Urodynamics 21: 261-274.
2. Madersbacher S, Alivizatos G, Nordling J, Sanz C R, Emberton M, et al. (2004) Eau 2004 guidelines on assessment, therapy and follow-up of men with lower urinary tract symptoms suggestive of benign prostatic obstruction (bph guidelines). European Urology 46: 547-554.
3. Nitti V W (2005) Pressure flow urodynamic studies: the gold standard for diagnosing bladder outlet obstruction. Rev Urol 7 Suppl 6: S14-21.
4. Pel J J M, van Mastrigt R (2002) Development of a low-cost flow meter to grade the maximum flow rate. Neurourology and Urodynamics 21: 48-54.
5, Addla S K, Marri R R, Daayana S L, Irwin P (2010) Avoid cruising on the uroflowmeter: Evaluation of cruising artifact on spinning disc flowmeters in an experimental setup. Neurourology and Urodynamics 29: 1301-1305.
6. Caffarel J, Robson W, Pickard R, Griffiths C, Drinnan M (2007) Flow measurements: Can several “wrongs” make a “right”? Neurourology and Urodynamics 26: 474-480.
7. Currie R J (1998) The streamtest cup: a new uroflow device. Urology 52: 1118-21.
8. delakosette J J M C H, Witjes W P J, Debruyne F M J, Kersten P L, Wijkstra H (1996) Improved reliability of uroflowmetry investigations: Results of a portable home-based uroflowmetry study. British Journal of Urology 78: 385-390.
9. Eggers J, Villemiaux E (2008) Physics of liquid jets. Reports on Progress in Physics 71: 1-79.
10. Eggers J, Dupont T F (1994) Drop formation in a one-dimensional approximation of the navierstokes equation. Journal of Fluid Mechanics 262: 205-221.
11. Lin S P, Reitz R D (1998) Drop and spray formation from a liquid jet. Annual Review of Fluid Mechanics 30: 85-105.
12. Papageorgiou D T (1995) On the breakup of viscous-liquid threads. Physics of Fluids 7: 1529-1544.
13, Rayleigh L (1878) On the instability of jets. Proceedings of the London Mathematical Society s1-10: 3.
14. Dietz H P (2007) Does bladder neck descent increase with age? International Urogynecology Journal 18: 665-669.
15. Steenkamp J W, Heyns C F, deKock M L S (1997) Internal urethrotomy versus dilation as treatment for male urethral strictures: A prospective, randomized comparison. Journal of Urology 157: 98-101.
16. Greenwell T J, Castle C, Andrich D E, MacDonald J T, Nicol D L, et al, (2004) Repeat urethrotomy and dilation for the treatment of urethral stricture are neither clinically effective nor cost-effective. Journal of Urology 172: 275-277.
17. Wiygul J, Babayan R K (2009) Watchful waiting in benign prostatic hyperplasia. Current Opinion in Urology 19: 3-6.
18. Neal D E (1997) Watchful waiting or drug therapy for benign prostatic hyperplasia? Lancet 350: 305-306.
19. Jonas U, Harter K (1996) Symptom scores, watchful waiting and prostate specific antigen levels in benign prostatic hyperplasia. Journal of Urology 156: 1040-1041.
20. Wasson J H, Reda D J, Bruskewitz R C, Elinson J. Keller A M, et al. (1995) A comparison of transurethral surgery with watchful waiting for moderate symptoms of benign prostatic hyperplasia. New England Journal of Medicine 332: 75-79.
21. Reynard J M, Peters T J, Lim C, Abrams P (1996) The value of multiple free-flow studies in men with lower urinary tract symptoms. British Journal of Urology 77: 813-818.
22. C-affarel J, Robson W, Pickard R, Newton D, Griffiths C, et al. (2006) Home uroflow device: Basic but more accurate than standard in-clinic uroflowmetry? Neurourology and Urodynamics 25: 632-633.
23. Bou R, Fall M, Walden M, Knutson T, Dahlstrand C (1999) Home uroflowmetry: improved accuracy in outflow assessment. Neurourol Urodyn
18: 25-32.
24. Golomb J, Lindner A, Siegel Y, Korczak D (1992) Variability and circadian changes in home uroflowmetry in patients with benign prostatic hyperplasia compared to normal controls. Journal of Urology 147: 1044-1047.
25. Wheeler A. P. S., Morad S. Buchholz N, Knight M M, The Shape of the Urine Stream—From Biophysics to Diagnostics PLOS ONE October 2012 |Volume 7 |Issue 10 |e47133
26. Wheeler A P S, “Physics on tap”January 2012 PHYSICS EDUCATION 47 (4) 403-408

Various alternative embodiments are also contemplated as being within the scope of the following claims, particularly pointing out and distinctly claiming the subject matter regarded as the invention.

1. A method of diagnosing a human patient with a uroligical disorder, the method comprising the steps of:
a. recording video and'or audio of the voiding urinary stream and/or the patient's face using one or more cameras; and
b. determining diagnostic information on the patient from the recorded video.

2. The method of claim 1 further comprising the step of storing the recorded video data in an electronic data storage device either concurrently with or subsequently to the step of recording video of the patient.

3. The method of claim 2 further comprising the step of transmitting the recorded video data to a location remote from the patient location either concurrently with or subsequently to the step of recording video of the patient.

4. The method of claim 1 in which the recorded video data are processed to create diagnostic information regarding the patient after recording video of the patient.

5. The method of claim 4 wherein the step of processing the recorded video data comprises determining urine flow rate from the recorded video data.

6. The method of claim 4 wherein the step of processing the recorded video data comprises determining meatal dilation from the recorded video data.

7. The method of claim 4 wherein the step of processing the recorded video data comprises determining the voiding flow versus time from the recorded video data.

8. The method of claim 4 wherein the step of processing the recorded video data comprises determining the voiding peak flow from the recorded video data.

9. The method of claim 4 wherein the step of processing the recorded video data comprises determining the voiding volume using recorded video from different view angles from the recorded video data.

10. The method of claim 4 further comprising the step of transmitting the processed recorded video data and/or diagnostic information to a location remote from the patient location.

11. The method of claim 1 wherein the step of recording video comprises recording video while the patient is in a standing position.

12. The method of claim 1 wherein the step of recording video comprises recording video while the patient is in a sitting position

13. The method of claim 1 further comprising the step of capturing the voiding urinary stream in a container.

14. The method of claim 13 further comprising the step of adding a container divider that reduces wave motion on the top of the captured urine stream.

15. The method of claim 13 further comprising the step of weighing the container to determine the volume and/or weight of the captured urine stream.

16. The method of claim 13 further comprising the step of recording video of an object floating on the captured urine stream.

17. A device for performing the method of claim 1.

18. A device for diagnosing a human patient with a urological disorder, the device comprising:
a. a booth including a number of walls;
b. at least one light mounted to the booth;
c. at least one camera mounted to the booth; and
d. at least one collection element disposed within the booth

19. The device of claim 18 further comprising a computing device operably connected to the at least one camera and configured for recording, storing and processing video obtained from the at least one camera and or microphone.

20. The device of claim 19 wherein the computing device is configured to transmit the recorded, stored and processed video from the at least one camera to a remote location.
